(19) United States
(12) Patent Application Publication
(10) Pub. No.: US 2015/0296960 A1
(43) Pub. Date: Oct. 22, 2015

(54) METHOD AND APPARATUS FOR CREATING ARTISTIC TEMPORARY DESIGNS

(71) Applicant: GLIMMER BODY ART LLC, Carson, CA (US)

(72) Inventor: Salustiano G. Samaco, III, Carson, CA (US)

(21) Appl. No.: 14/531,690

(22) Filed: Nov. 3, 2014

Related U.S. Application Data
(60) Provisional application No. 61/898,659, filed on Nov. 1, 2013.

(51) Int. Cl. A45D 40/30; A61K 8/81; A61K 8/34; A61Q 1/02

(52) U.S. Cl. CPC A45D 40/30; A61Q 1/025; A61K 8/8147; A61K 8/8164; A61K 8/345; A61K 2800/42; A61K 2800/30; A61K 2800/72

(57) ABSTRACT

A temporary body art design and a method of applying the same. The temporary design is formed on a screen and defined by transmissive portions and blocking portions. A first adhesive material is applied to a first side of the screen and a release membrane extends over the first adhesive material. To apply the design to an individual's skin, the screen is placed on a region of the individual's skin, an adhesive, material or colorant is applied over the transmissive portions of screen and pass therethrough to form the design on the individual's skin. If an adhesive is applied to the screen, a material or colorant can then be applied over the adhesive upon removal of the screen from the skin.

### CROSS-REFERENCE TO RELATED APPLICATION

This patent application claims benefit under 35 U.S.C. §119 to U.S. Provisional Patent Application No. 61/898,659, filed Nov. 1, 2013, which is hereby incorporated by reference in its entirety as part of the present disclosure.

### FIELD OF THE INVENTION

The present invention relates broadly to body art and more particularly to temporary body art and a method of applying the same.

### BACKGROUND OF THE INVENTION

The creation of artistic designs on a body, commonly referred to as “body art,” is a tradition that dates back centuries. Generally, body art is either permanent (e.g., tattooing) or temporary (e.g., designs that can be removed at will or designs that will fade away over time). Temporary designs are often preferred over permanent designs because they are not painful and can be easily and quickly applied. Additionally, temporary designs are removable and, in many situations, they can be modified at will.

One known method of creating and applying a temporary design includes free form painting using a paint brush, crayons, or similar instruments. In many instances, designs created in this manner can be complex, time consuming, and require the expertise of a skilled individual to create such designs.

Another known method of creating and applying a temporary design involves creating a sticker by printing a desired design on one side of paper and applying an adhesive backing on the other side to permit the design to be kept in place. However, the size of a sticker is very limited because designs that require printing on a large sticker are commonly uncomfortable to wear and in many instances, stickers are not ornate or attractive to wear. Additionally, stickers are not designed for an extended period of use and can easily fall off of a user's skin, especially if the user's skin becomes wet or the user perspires.

Yet another known method of creating and applying a temporary design is to use a stencil (i.e., a die), which is commonly a brittle sheet of Mylar or other vinyl that includes a hole defining an outline of the design. To apply the stencil, it is commonly placed against skin and paint or other coloring material is applied to one side of the stencil. The coloring material passes through the hole onto an individual's skin. The stencil is then removed, leaving behind a design. However, because these designs are only formed through holes, this technique is only practical for simple design. To create an ornate design several large holes with “floats” or “islands” (e.g., portions formed independent of each other creating spaces between the portions) therebetween must be made in the stencil with many or all portions of the stencil disconnected. To apply such a stencil design to an individual's skin, a small tool is required, such as a tweezer, to move the floaters into their correct position and even after being positioned, the floaters have a tendency to move, distorting the design.

Stencils have also been used to apply make-up. These stencils are commonly non-pliable, Mylar material that can be used to replicate designs on an area of skin (e.g., lips, eyebrows) with a pen, a pencil, or other coloring device. However, here only simple stencils can be used because the application is only to a small region of a user's skin and any intricate design would require floaters that would be very challenging to maintain in a desired location.

Yet a further method of using stencils is to apply a pattern or design to a user's eye lids or lips to create a look, such as an ombre. To create an ombre, gradient coloring is required and achieved by applying lipstick/lip stain/lip gloss of one shading on the center of the stencil and then different shading is applied to the outer perimeter of the stencil. However, because of the intricacy of the design and location of application (e.g., lips, eyebrows), a steady hand of a make-up artist is required to achieve the desired look. As such, a typical consumer regularly struggles only to fail to achieve the desired look after exhaustive and time-consuming attempts.

### SUMMARY OF THE INVENTION

The present invention is directed to temporary body art and a method of applying the same.

In an embodiment, the present invention is directed to a stencil comprising a screen that includes a design applied thereon forming transmissive portions and blocking portions, a first adhesive material applied to a first side of the screen and a release membrane extending over the first adhesive material. In an embodiment, the stencil can be adapted to contour to a portion of the skin including at least one of an individual's lips or eyelids.

In an embodiment, the screen can be a thin, malleable sheet comprised of tightly-woven mesh material and the mesh material of the screen at the transmissive portions can permit a second adhesive material, colorant or other substance to pass therethrough. The blocking portions of the screen can prohibit any substance to pass therethrough.

In an embodiment, the first adhesive layer is malleable and includes a composition that is suitable for application on skin.

In an embodiment, the present invention is directed to a method of applying a temporary design to skin comprising the following steps: selecting a stencil design that includes transmissive portions and blocking portions, peeling a release membrane from a first side of the stencil design to expose a first adhesive layer, applying the first side of the stencil design to skin, applying a material onto a second side of the stencil such that the material passes through the transmissive portions defining the stencil design and onto the skin, removing the stencil design from the skin and applying at least one material or colorant to the second adhesive layer forming the stencil design on the skin. The at least one material or colorant can be at least one of glitter, powder, liquid or removable paint.

In an embodiment, the method can further comprise the step of preparing the skin by applying a base layer of antiseptic solution to the skin prior to applying the first side of the stencil design to the skin.

In an embodiment, the method can further comprise the step of drying the second adhesive material prior to applying the at least one material or colorant thereon.

In an embodiment, the second adhesive layer is a latex free, hypo-allergenic body glue. The body glue can comprise following composition: 39-41% acrylic polymer; 14-16% polyvinyl acetate; 1-3% butylene glycol; and water.

In an embodiment, the method can further comprise the step of applying a second layer of the second adhesive material to regions on or near the stencil design on the skin and subsequently applying at least one decorative element to the skin where the second layer of the second adhesive material is applied.

In an embodiment, the present invention is directed to a method of applying a temporary design to skin comprising the following steps: selecting a stencil design that includes transmissive portions, blocking portions, a first adhesive layer and a release membrane extending over the first adhesive layer, peeling the release membrane from a first side of the stencil design to expose the first adhesive layer, applying the first side of the stencil design to skin, applying at least one first material or colorant onto a second side of the stencil design such that the at least one material or colorant passes through the transmissive portions defining the design and onto the skin and removing the stencil from the skin. The at least one material or colorant can be at least one of glitter, powder, liquid or removable paint. In an embodiment, the method can further comprise the step of applying at least one second material or colorant is applied over the at least one first material or colorant.

### BRIEF DESCRIPTION OF THE DRAWINGS

FIGS. 1A-1E are exemplary embodiments of known stencil designs;

FIGS. 2A-2I are stencil designs constructed in accordance with embodiments of the present invention;

FIG. 3 is a side view of a stencil constructed in accordance with an embodiment of the present invention;

FIG. 4 is a flow chart outlining embodiments of methods of making and applying stencils of the present invention;

FIG. 5A-5C are embodiments stencils of the present invention for the application of coloring to eyelids, lips or other extremities of the body;

FIG. 6 is a rendering of an individual's face illustrating effects that can be achieved by applying make-up using a stencil in accordance with an embodiment of the present invention; and

FIGS. 7A-7D are photographic images of make-up applied using embodiments of stencils of the present invention.

### DETAILED DESCRIPTION OF EMBODIMENTS OF THE PRESENT INVENTION

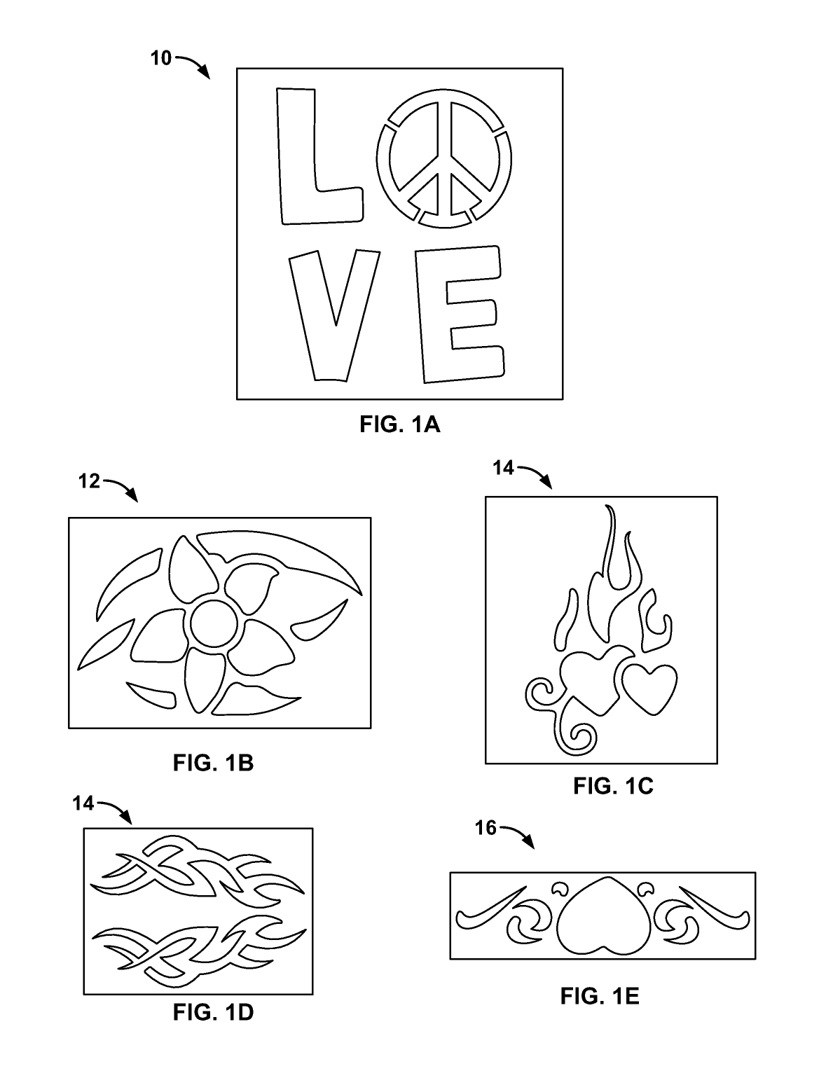

With reference now to the drawings, by way of example, FIGS. 1A through 1E illustrate relatively simplistic existing stencil designs 10, 12, 14, 16, 18. As shown, some of the designs 10, 12, 14, 16, 18 require connectors to link all aspects of a given one of the designs 10, 12, 14, 16, 18 to each other and/or aspects of some of the designs 10, 12, 14, 16, 18 are separated by “floats.” In FIG. 1A, for example, the letter “O” in the word “LOVE” is formed with connectors that attach the central portion of the letter with the rest of the background. In FIG. 1B, for example, a flower and decorative features surrounding the flower are comprised of floats that regularly require positioning using a small tool, such as a tweezer, and have a tendency to move even after positioning after being released by a tool.

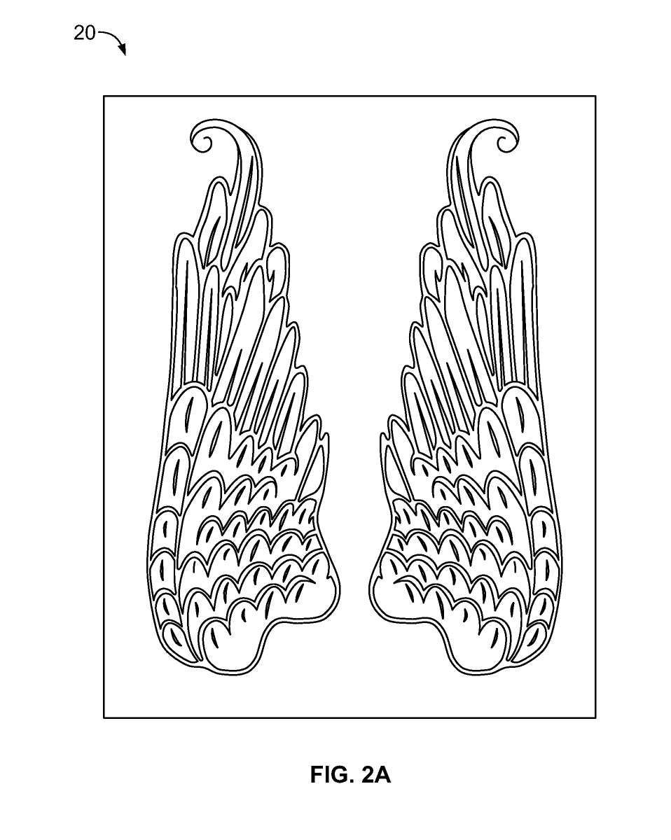

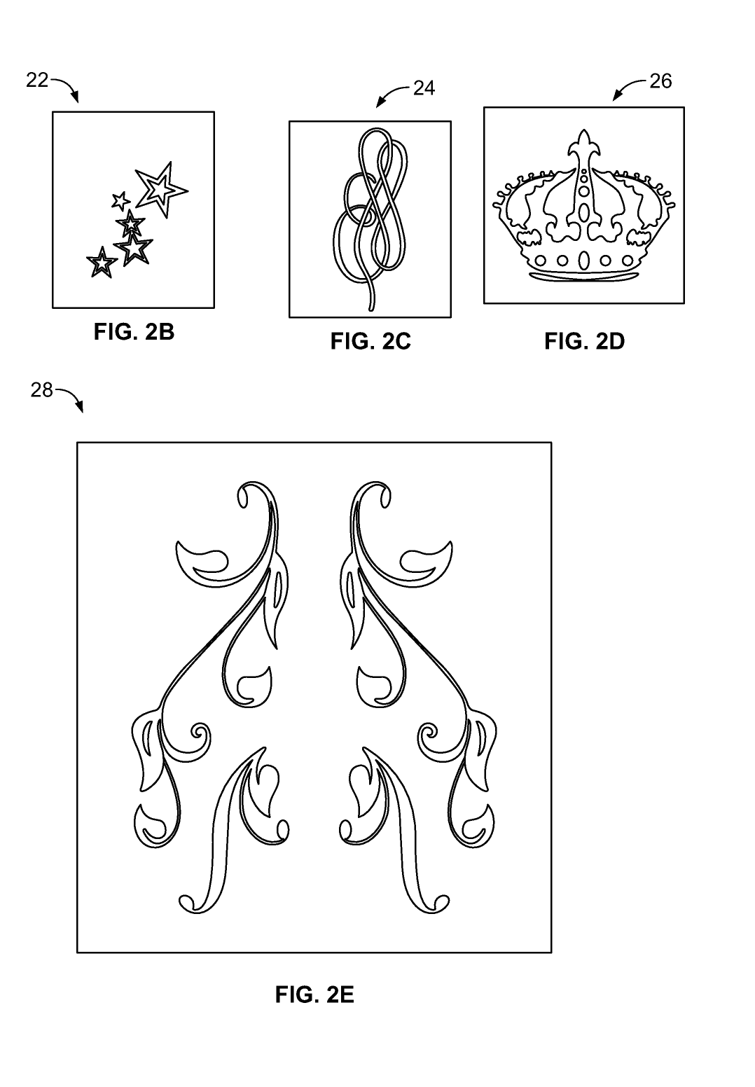

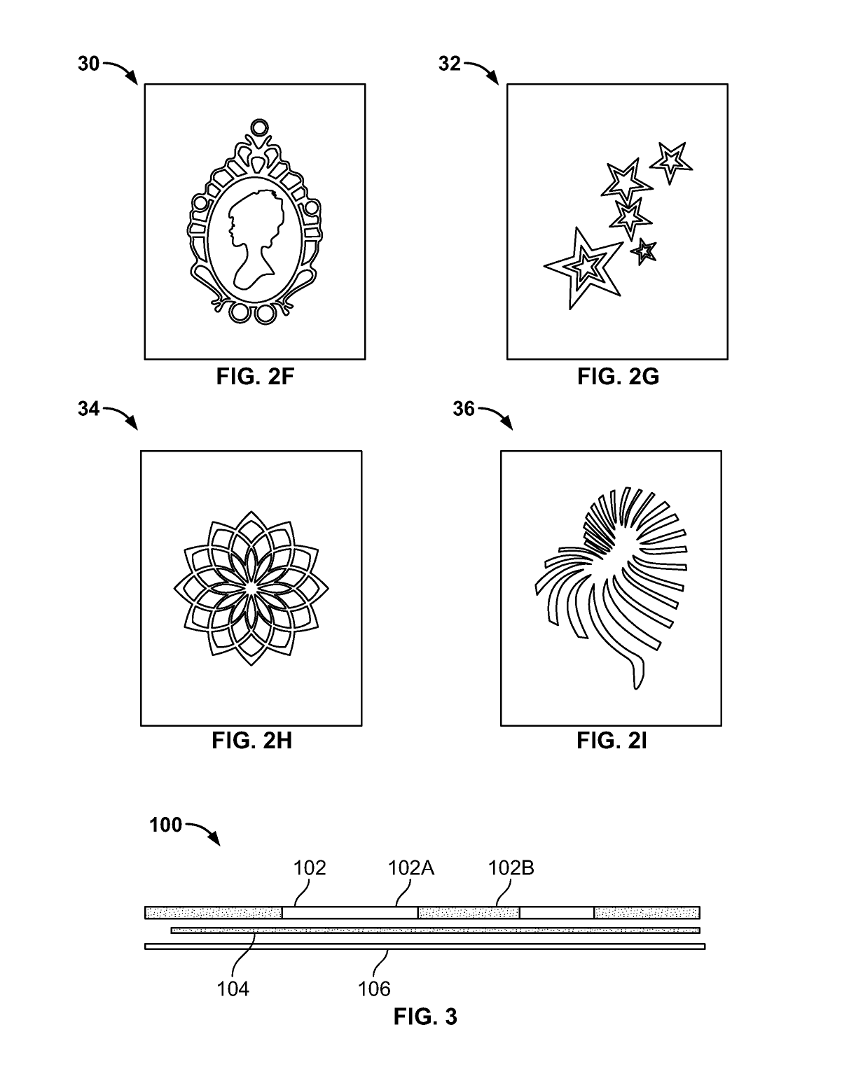

Embodiments and features embodying the principles and concepts of the present invention will be described are depicted in FIGS. 2A through 7C.

FIGS. 2A-2I show embodiments photo-realistic detailed designs 20, 22, 24, 26, 28, 30, 32, 34, 36 of the present invention that are each imprinted on a tightly-woven screen made of a mesh material. As a result of being printed on a screen, the designs 20, 22, 24, 26, 28, 30, 32, 34, 36 can be extremely ornate and include floats or islands (e.g., FIGS. 2B, 2G) as the placement of the floats or islands do not require individual arrangement because they are part of a single screen.

FIG. 3 illustrates a side view of an embodiment of a stencil 100 that includes a design in accordance with the present invention. As shown, the stencil 100 includes a thin, malleable sheet 102 comprised of a tightly-woven mesh screen that can be attached and easily contour to a portion of a body. The sheet 102 includes transmissive portions 102A and blocking portions 102B that form a design. A first adhesive layer 104, for applying the sheet 102 to the skin of a user, is deposited on one side of the sheet 102. The first adhesive layer 104 is protected by a release membrane 106 that is generally coextensive with and covers the sheet 102. In an embodiment, the first adhesive layer 104 includes at least one edge 104A that does not extend as far as the release membrane 106 to permit removal of the release membrane 106 easily from the sheet 102 and the first adhesive layer 104. In an embodiment, the stencil 100 can include a covering layer (not shown) arranged over a side of the sheet 102, opposite the side of the sheet 102, to protect the design. The covering layer can include at least one edge that does not extend as far as the outer periphery of the sheet 102 to permit removal of the release membrane 106 easily from the sheet 102.

In an embodiment, the first adhesive layer 104 is malleable and made of a material composition that is suitable for application on a user's skin (e.g., similar to an adhesive used in conjunction with a bandage). However, the first adhesive layer 104 should not include a material composition such that it can be removed without an application of force or have a tackiness such that it requires a significant force that could cause pain or injury to a user to remove. The first adhesive layer 104, unlike vinyl or Mylar, is not brittle, can be contoured to curves of skin and can maintain the shape of a detailed design without the manifestation of floats, islands, or connectors. This is because the screen 102 overlay ensures that any floats, islands, or connectors or other details are not comprised and/or left on the skin after the stencil 102 is removed.

In an embodiment, the sheet 102 is made using silk-screening techniques with the transmissive portions 102A including a fine mesh material made of thin fibers and the blocking portions 102B made by imprinting or otherwise depositing an ink or a similar material thereon. Liquids and pastes can be applied to the sheet 102 and are permitted to pass through the transmissive portions 102A, but not the blocking portions 102B of the sheet 102.

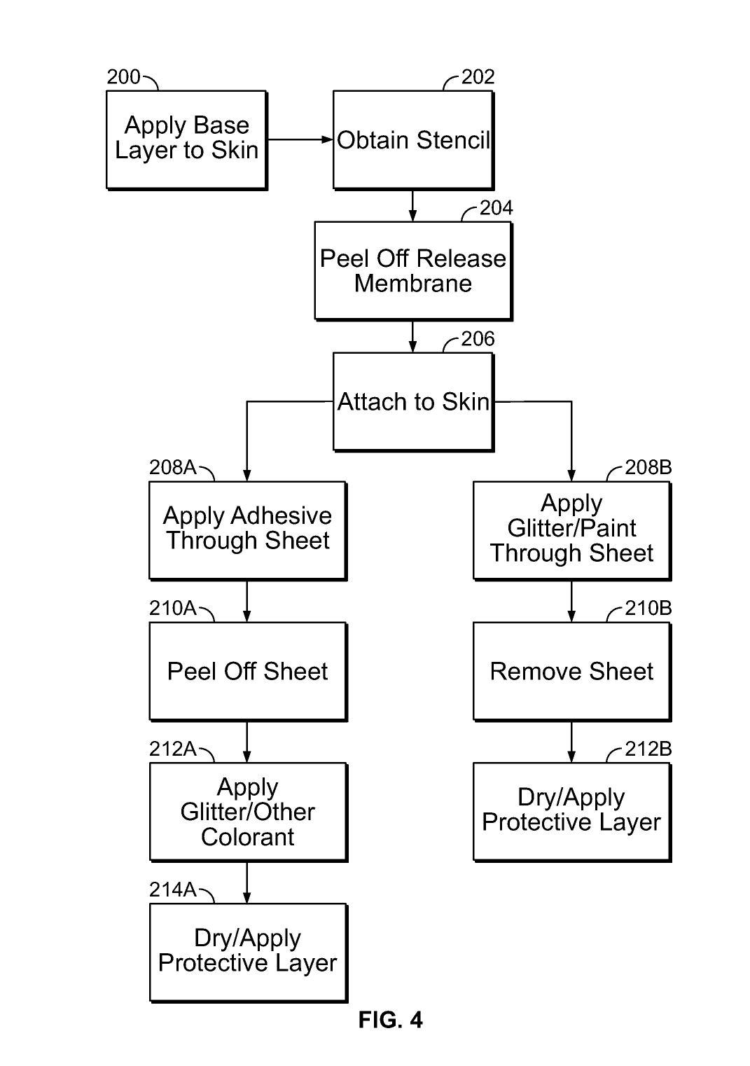

FIG. 4 illustrates a flow chart outline embodiments to apply the stencil 102 and associated artistic design on an individual's skin. Depending on the complexity of the design, a treatment applying a stencil 102 can last anywhere from about 10 to 50 minutes.

In an embodiment, first, as shown at step 200, the individual's skin is prepared for the application of the stencil 102 by applying a base layer to the individual's skin and then allowing the base layer to dry. The base layer can include, rubbing alcohol, a soap based solution or any other antiseptic or cleaning product suitable for use with a human body to remove any oil or other residue from the area of application. Next, in step 202, a desired stencil 102 design is selected/obtained. The release membrane 16 is then peeled from the stencil 102 (step 204) and in step 206 the sheet 102 is pressed against an individual's skin to secure the sheet 102 by the first adhesive layer 104 to an individual. If a protective sheet is affixed to the sheet 102, it is then removed.

In an embodiment, as indicated at step 208A, a second adhesive layer is then applied using an applicator such as a paint brush, roller or spraying device to the outer surface of the sheet 102 while the sheet 102 is affixed to an individual. The second adhesive layer must be thin enough so that it can pass through the screen or mesh forming the transmissive portions 102A to contact the individual's skin. In an embodiment, the second adhesive layer is a latex free hypo-allergenic body glue. The glue can includes the following composition: 39-41% acrylic polymer; 14-16% polyvinyl acetate; 1-3% butylene glycol; and water. An advantage of this glue is that even though it is water-based, it will not freeze when transported by air.

Next, in step 210A, the sheet 102 is peeled off of the skin leaving the second adhesive layer formed in a pattern defined by the transmissive areas 102A. In step 212A, after the second adhesive layer dries (turns clear), a liquid, fine powder, glitter, removable paint or other matte colorant that can last up to about 7 days is applied over the second adhesive layer on the user's skin (no adhesive associated with powder, glitter, etc.). The excessive colorant in step 214A is then removed from the skin and the remaining colorant in step 216A is allowed to dry thereby, forming a new body art.

In an embodiment, the second adhesive layer can be further applied on or near the design allowing for the inclusion of crystals, gemstones, or other decorative elements to further enhance the design. In an embodiment, a light protective layer may be applied to the skin to protect the body art.

In another embodiment, after completing steps 200 through 206 described above, at step 208B, a cosmetic liquid, powder, glitter, paint or other colorant is applied directly to the sheet 102. The sheet 102 is then removed (step 210B) leaving the colorant in a pattern defined by the transmissive portions 102A of the design. The colorant at step 212B is then dried. In an embodiment, a further application of the second adhesive layer can be applied on or near the design to further apply crystals, gemstones or other decorative elements to the design to further enhance the design. In an embodiment, a light protective layer may be applied to the skin to protect the body art.

In an embodiment, after applying a base layer to the user's skin and allowing the base layer to dry to clean the area of skin, paint or other colorant can be applied directly to the user's skin by free hand or using another stencil. The stencil 100 can then be applied to a user's skin and a colorant that is different from that applied directly to the user's skin can be applied onto the stencil and passes through the screen or mesh of the transmissive portion 102A to generate a complex, multi-color design.

In an embodiment, the colorant can be a black ink that includes the following composition: 44.30% water; 18.90% polyacrylic emulsion; 13.70% calcium carbonate (CL 77220); 10.50% polyvinylpyrrolidone; −3.50% glycerol; −3.20% 1, 3-Butylene glycol; −2.10% fumed silica; −1.40% pigment black 11 (CL 77499); −0.70% FD&C Yellow 5 AL lake (CL 19140:1); −0.70% FD&C Blue No. 1 AL lake (CL 42090:2); −0.68% phenoxyethanol; −0.16% methylparaben; −0.05% ethylparaben; −0.05% butylparaben; −0.03% isobutylparaben; and −0.03% propylparaben. As an alternative to black ink, by changing the colorants in the above composition, the ink can be red, PMS 478C or another color as well.

Stencils described herein are usable not only for body art, but also for applying make-up. The application of make-up can be vastly improved using a stencil design of the present invention to allow individuals to create detailed artwork on their skin as well as their lips, eye lids or other areas of their body at an affordable price and without relying on the assistance of skilled professionals. For example, to achieve the ombre effect described above, consumers need only to affix a stencil shaped for an individual's lips by a first adhesive layer to their lips and then apply a complimentary color on top of the stencil. The tackiness of the first adhesive layer allows use of both hands in creating the ombre effect, which in turn cuts down the application time from 10 to 2 minutes.

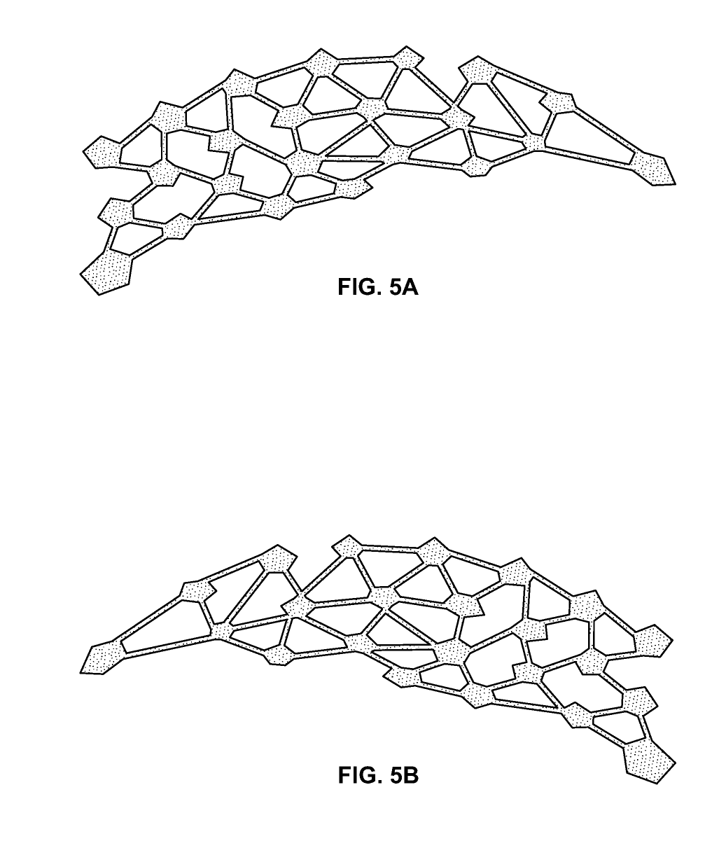

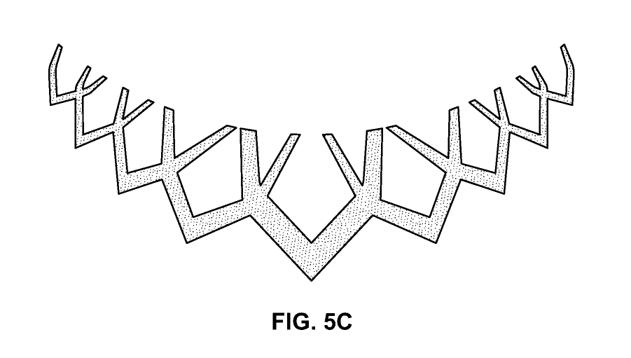

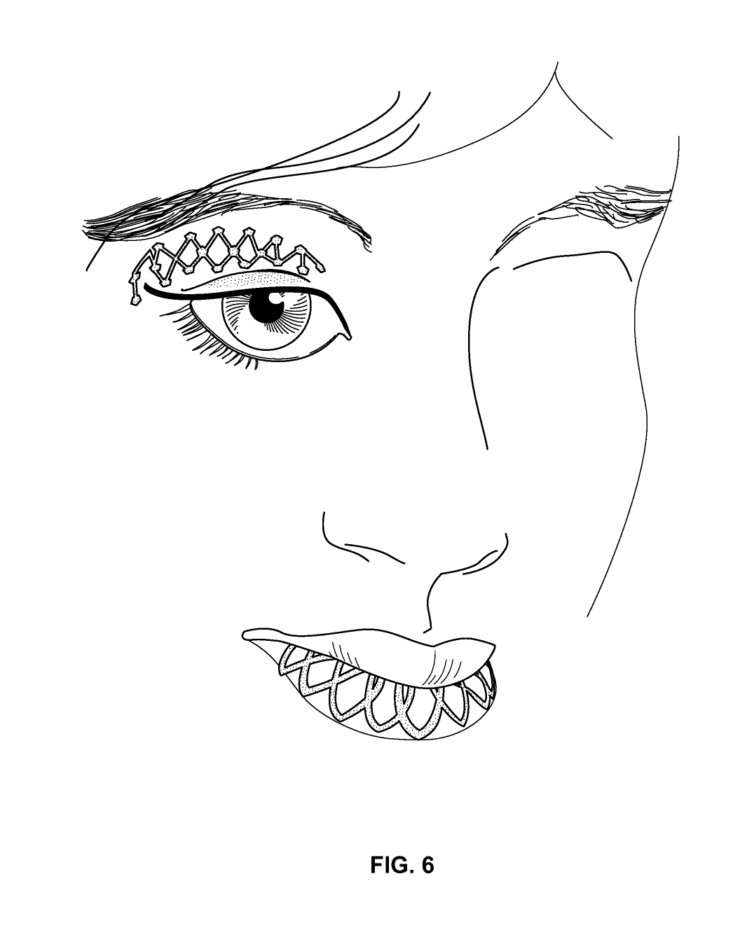

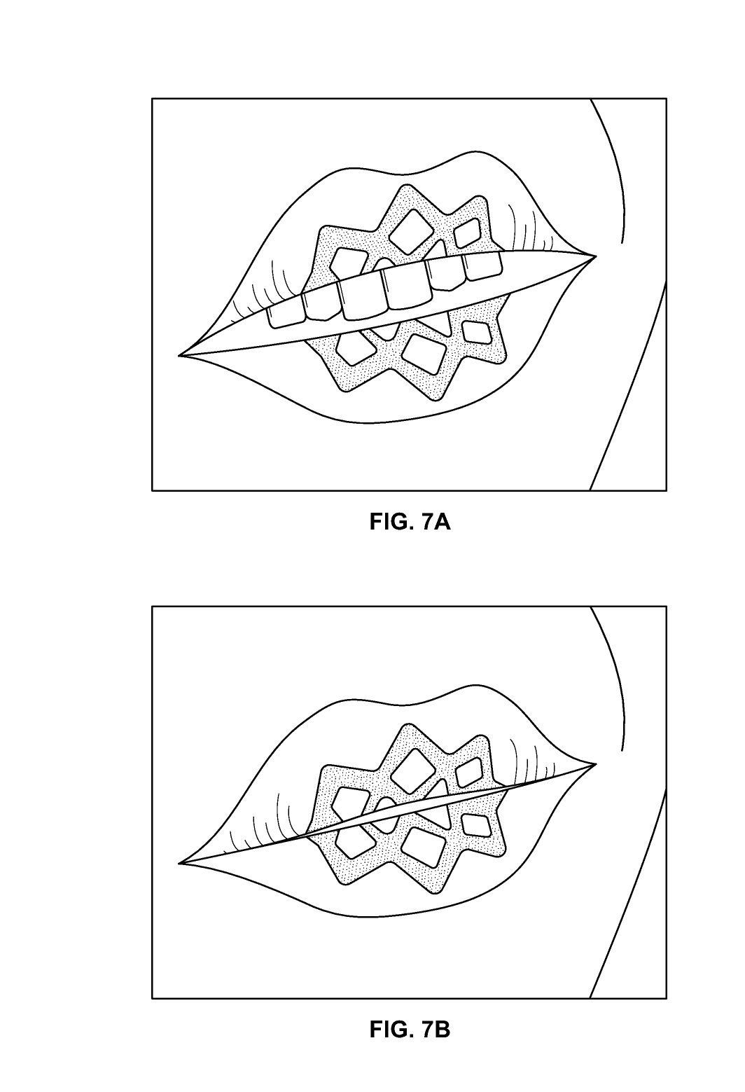

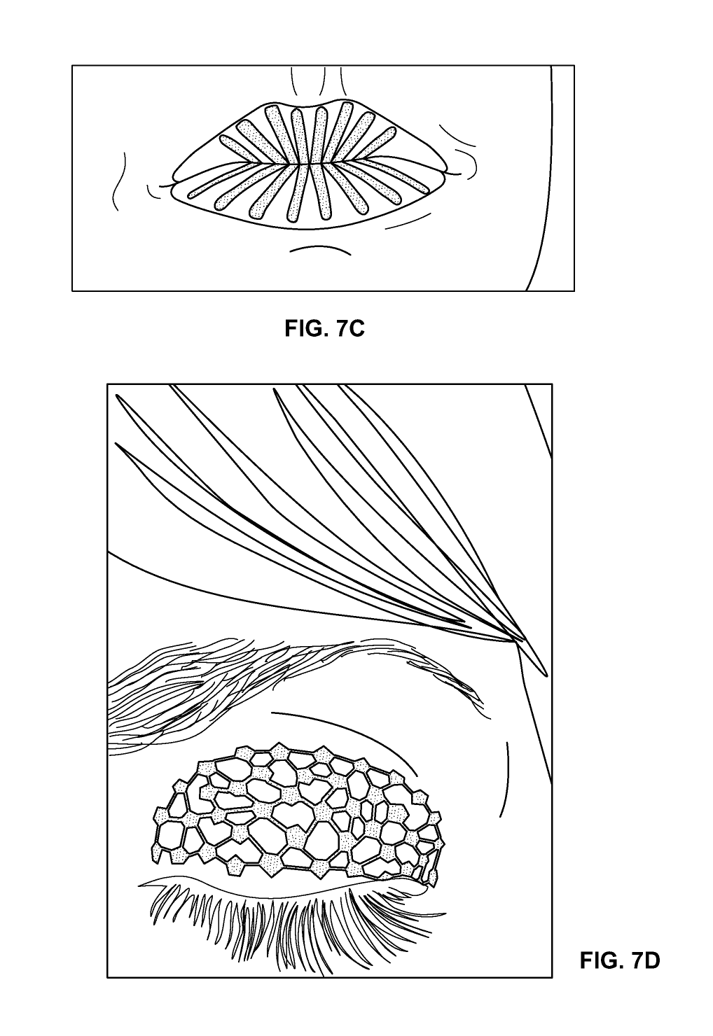

Embodiments of stencil designs that can be applied to an individual's eye lids or lips are shown in FIGS. 5A through 5C. FIG. 6 is a rendering of an individual's face illustrating effects that can be achieved by applying make-up using a stencil in accordance with an embodiment of the present invention. FIGS. 7A through 7C are photographic images of designs applied to an individual's lips eyes and FIG. 7D is a photographic image of a design applied to a user's lips accordance with an embodiment described above.

Although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure.

What is claimed is:

1. A method of applying a temporary design to skin, comprising the following steps:
selecting a stencil design that includes transmissive portions and blocking portions;
peeling a release membrane from a first side of the stencil design to expose a first adhesive layer;
applying the first side of the stencil design to skin;
applying a material onto a second side of the stencil such that the material passes through the transmissive portions defining the stencil design and onto the skin;
removing the stencil design from the skin; and
applying at least one material or colorant to the second adhesive layer forming the stencil design on the skin.

2. The method of claim 1, further comprising the step of preparing the skin by applying a base layer of antiseptic solution to the skin prior to applying the first side of the stencil design to the skin.

3. The method of claim 1, further comprising the step of drying the second adhesive material prior to applying the at least one material or colorant thereon.

4. The method of claim 1, wherein the second adhesive layer is a latex free, hypo-allergenic body glue.

5. The method of claim 4, wherein the body glue comprises following composition: 39-41% acrylic polymer; 14-16% polyvinyl acetate; 1-3% butylene glycol; and water.

6. The method of claim 1, wherein the at least one material or colorant is at least one of glitter, powder, liquid or removable paint.

7. The method of claim 1, further comprising the step of applying a second layer of the second adhesive material to regions on or near the stencil design on the skin and subsequently applying at least one decorative element to the skin where the second layer of the second adhesive material is applied.

8. A method of applying a temporary design to skin, comprising the following steps:
selecting a stencil design that includes transmissive portions, blocking portions, a first adhesive layer and a release membrane extending over the first adhesive layer;
peeling the release membrane from a first side of the stencil design to expose the first adhesive layer;
applying the first side of the stencil design to skin;
applying at least one first material or colorant onto a second side of the stencil design such that the at least one first material or colorant passes through the transmissive portions defining the design and onto the skin; and
removing the stencil from the skin.

9. The method of claim 8, further comprising the step of applying at least one second material or colorant is applied over the at least one first material or colorant.

10. The method of claim 8, wherein the at least one first material or colorant and the at least one second material or colorant is at least one of glitter, powder, liquid or removable paint.

11. The method of claim 8, further comprising the step of preparing the skin by applying a base layer of antiseptic solution to the skin prior to applying the first side of the stencil design to the skin.

12. The method of claim 8, further comprising the step of applying a second adhesive material to the at least one material or colorant on the skin and subsequently applying at least one decorative element to the skin where the second layer of the second adhesive material is applied.

13. The method of claim 12, wherein the second adhesive layer is a latex free, hypo-allergenic body glue.

14. The method of claim 13, wherein the body glue comprises following composition: 39-41% acrylic polymer; 14-16% polyvinyl acetate; 1-3% butylene glycol; and water.
